(19) United States
(12) Patent Application Publication
(10) Pub. No.: US 2025/0140347 A1
(43) Pub. Date: May 1, 2025

(54) METAGENOMIC FILTERING AND USING THE MICROBIAL SIGNATURES TO AUTHENTICATE FOOD RAW MATERIALS

(71) Applicant: Mars, Incorporated, McLean, VA (US)

(72) Inventors: Balasubramanian GANESAN, Vancouver, WA (US); Robert C. BAKER, Geylang (SG); David F. CREAN, Harringworth (GB)

(73) Assignee: Mars, Incorporated, McLean, VA (US)

(21) Appl. No.: 18/683,321

(22) PCT Filed: Aug. 15, 2022
(86) PCT No.: PCT/US2022/074959

§ 371 Date: Feb. 13, 2024

Related U.S. Application Data
(60) Provisional application No. 63/234,093, filed on Aug. 17, 2021.

(51) Int. Cl. G16B 30/00; C12Q 1/689

(52) U.S. Cl. CPC G16B 30/00; C12Q 1/689; C12Q 2600/158

(57) ABSTRACT

Methods and systems for authenticating or identifying a food source for a food product are disclosed herein. Also disclosed herein are methods and systems for detecting a contaminant in a food product. The methods comprise obtaining sequence data for a plurality of nucleic acid sequences present in a food product, identifying one or more microbial signatures, wherein the one or more microbial signatures correspond to one or more microbes present in the food product, and determining whether the one or more microbial signatures correspond to one or more microbes associated with a particular food source or a particular contaminant.

### CROSS REFERENCE TO RELATED APPLICATIONS

This application is a U.S. National stage application under 35 U.S.C. § 371 of International Application No. PCT/US2022/074959, filed Aug. 15, 2022, which claims priority to U.S. Provisional Patent Application No. 63/234,093, filed Aug. 17, 2021. The entire contents of each priority application is incorporated herein by reference.

### FIELD OF THE DISCLOSURE

This disclosure relates to methods of authentication of food raw materials and detection of food contaminants using microbiome analysis and metagenomics filtering.

### BACKGROUND OF THE DISCLOSURE

The authenticity of raw materials used for food production is a subject of great concern for food producers, consumers, and food authorities alike. The quality and cost of a particular food product can vary greatly based on its source or supplier. For example, a particular raw material may have vastly different quality, flavor, and/or cost based on the geographic origin, growing conditions, breed or variety, production method, or animal feeding regime used as the source. For food producers, properly authenticating the raw materials used in their products is important to maintaining consistency and confidence in their food products.

Lack of food authenticity can lead to both economic and public safety concerns. The presence of contaminants or erroneously labeled food sources in a food product can compromise quality, safety, and confidence in the company products. Lack of authenticity due to intentional or un-intentional adulteration or mislabeling of food products is closely monitored by food authorities, as it may represent commercial fraud or compromised food safety. Food authentication and, in particular, identification of components of food products is therefore important in controlling food quality and safety and in protecting consumers.

Efficient and reliable techniques for determining food authenticity are required. Some existing food authentication methods have relied on targeted detection of food raw materials by chemical or biological means, such as through qPCR, chromatography, or enzymatic analysis. High throughput methods for analysis of nucleic acids or proteins from food raw materials for direct identification, for example, using sequencing or proteomics, have also been developed. However, these methods require that a specific component or source is present at the time of analysis. Thus, if the specific component or source material are removed, the source may fail to be detected.

Accordingly, there is a need for high-throughput end-to-end methods for food source authentication and trace-back that do not rely solely in direct identification of food raw materials.

### SUMMARY OF THE INVENTION

Disclosed herein are systems and methods for authenticating or identifying a source for a food product. These methods may also be used for detecting contaminants in a food production chain.

In one aspect, disclosed herein is a method for authenticating a source for a food product comprising: obtaining sequence data for a plurality of nucleic acid sequences present in a food product; identifying one or more microbial signatures in the sequence data; and authenticating a source for the food product by determining whether the one or more microbial signatures correspond to one or more microbial signatures associated with a particular source for the food product, wherein the food product is authenticated if the one or more microbial signatures correspond to the one or more microbial signatures associated with a particular source for the food product.

In another aspect, disclosed herein is a method for identifying a source for a food product comprising: obtaining sequence data for a plurality of nucleic acid sequences present in a food product; identifying one or more microbial signatures in the sequence data; and identifying a source for the food product by determining whether the one or more microbial signatures correspond to one or more microbial signatures associated with a particular source for a food product, wherein the food source is identified if the one or more microbial signatures correspond to the one or more microbial signatures associated with a particular source for a food product.

In yet another aspect, disclosed herein is a method for detecting a contaminant in a food production chain, comprising: obtaining sequence data for a plurality of nucleic acid sequences present in a food product; identifying one or more microbial signatures in the sequence data; and detecting a contaminant in the food product by determining whether the one or more microbial signatures correspond to one or more microbial signatures associated with a particular contaminant, wherein the contaminant is detected if the one or more microbial signatures correspond to the one or more microbial signatures associated with a particular contaminant.

The one or more microbial signatures may correspond to one or more microbes present in the food product. The one or more microbial signatures may correspond to the genus taxonomy of one or more microbes present in the food product. The one or more microbial signatures may correspond to the species, serotype or strain taxonomy of one or more microbes present in the food product. The one or more microbial signatures may correspond to the kingdom, phyla, or class taxonomy of one or more microbes present in the food product. In some instances, the one or more microbial signatures may correspond to the quantitative or relative level of the one or more microbes present in the food product.

The one or more microbial signatures may correspond to one or more microbes associated with a single source or a single contaminant. The one or more microbial signatures may correspond to one or more microbes associated with two or more sources or two or more contaminants.

The source or the contaminant may be of animal origin. The animal origin may be egg, poultry meal, fish meal, or bone meal. The animal origin may also be various meats of animals such as canine, feline, equine, swine, bovine, ovine, or comparable sources, including game meat. The source or the contaminant may be of plant or fungal origin. The plant origin may be corn, rice, wheat, groundnut or peanut, or any variety thereof, or an equivalent domesticated or wild crop. The fungal origin may be of various yeasts and molds, including various mushrooms. The source or the contaminant may correspond to a particular geographical region.

The one or more microbes may be selected from the group consisting of bacteria, viruses, archaea, and eukaryotic microorganisms. The one or more microbes may belong to a genus taxonomy selected from the group consisting of: Parageobacillus, Blautia, Aliivibrio, Porphyrobacter, Shigella, Aneurinibacillus, Anaerostipes, Photobacterium, Erythrobacter, Rathayibacter, Butyrivibrio, Tyzzerella, Grimontia, Dechloromonas, Leifsonia, Coprothermobacter, Intestinimonas, Pseudoalteromonas, Pseudarthrobacter, Arthrobacter, Megasphaera, Ethanoligenens, Alteromonas, Isoptericola, Micrococcus, Eubacterium, Colwellia, Cellulomonas, Thermus, Oscillibacter, Yersinia, Nocardia, Meiothermus, Weissella, Edwardsiella, Gordonia, Rahnella, Murdochiella, Oceanimonas, Propionibacterium, Azotobacter, Eggerthella, Marinomonas, Tessaracoccus, Caulobacter, Adlercreutzia, Halomonas, Pimelobacter, Fibrobacter, Gordonibacter, Methylophaga, Actinoplanes, Fervidobacterium, Obesumbacterium, Brucella, Listeria, Methanobrevibacter, Plesiomonas, Caldanaerobacter, Deinococcus, Methanosarcina, Gallibacterium, Synechococcus, Spirosoma, Thioploca, Calothrix, Helicobacter, Thermotoga, Janthinobacterium, Nonlabens, Barnesiella, Fusobacterium, Ornithobacterium, lyobacter, Akkermansia, Thermodesulfobacterium, Cloacibacillus, Theileria, Gyrovirus, T7virus, T4virus, Alpharetrovirus, Sp8virus, Acidaminococcus, Altererythrobacter, Comamonas, Arcobacter, Aeromicrobium, Pediococcus, Proteus, Alistipes, Azospira, Geobacillus, Geoalkalibacter, Agrobacterium, Vibrio, Christensenella, Bosea, Kurthia, Hafnia, Alcaligenes, Clostridioides, Novosphingobium, Oblitimonas, Morganella, Amycolatopsis, Odoribacter, Pseudoxanthomonas, Negativicoccus, Aureimonas, Olsenella, Psychrobacter, Paenibacillus, Brachybacterium, Parabacteroides, Shewanella, Providencia, Brevibacterium, Roseburia, Candida, Ruminococcus, Caulimovirus, Selenomonas, Clavibacter, Treponema, Curtobacterium, Turicibacter, Erwinia, Frondihabitans, Hymenobacter, Kineococcus, Kluyveromyces, Massilia, Methylobacterium, Microbacterium, Nocardioides, Ochrobactrum, Pseudonocardia, Rhizobium, Saccharopolyspora, Sanguibacter, Shinella, Sphingobacterium, Sugiyamaella, Chryseobacterium, Aeromonas, Achromobacter, Blastomonas, Pantoea, Delftia, Anoxybacillus, Bordetella, Mycobacterium, Bacteroides, Brevundimonas, Rhodococcus, Bifidobacterium, Kosakonia, Streptomyces, Desulfovibrio, Sphingobium, Thermothelomyces, Flavonifractor, Sphingomonas, Thielavia, Lachnoclostridium, Sphingopyxis, Macrococcus, Cupriavidus, Moraxella, Prevotella, Ruminiclostridium, Bradyrhizobium, Campylobacter, Clostridium, Stenotrophomonas, Burkholderia, Cutibacterium, Xanthomonas, Serratia, Escherichia, Staphylococcus, Streptococcus, Variovorax, Acidovorax, Acinetobacter, Bacillus, Citrobacter, Corynebacterium, Enterobacter, Enterococcus, Klebsiella, Lactobacillus, Lactococcus, Pseudomonas, Raoultella, and Salmonella.

The step of obtaining the sequence data may comprise preparing a sequencing library. Obtaining the sequence data may comprise next generation sequencing or microarray analysis. The plurality of nucleic acid sequences may be DNA or RNA sequences. Sequences corresponding to the food product may be filtered from the sequence data prior to identifying the one or more microbial signatures.

The step of identifying the one or more microbial signatures may comprise comparing the sequence data against one or more databases of microbial nucleic acid sequences.

The step of determining whether the one or more microbial signatures correspond to one or more microbial signatures associated with a particular source or with a particular contaminant for the food product may include comparing the one or more microbial signatures against one or more databases of microbial signatures associated with the particular source; or comparing the one or more microbial signatures against one or more databases of microbial signatures associated with the particular contaminant.

Any of the methods disclosed herein may be performed at two or more points in a food production chain of the food product. The methods further comprise tracing the source or the contaminant to a particular supplier of the food product.

In another aspect, disclosed herein is a system for authenticating a source for a food product, comprising one or more processors and a memory comprising instructions executable by the one or more processors that, when executed by the one or more processors, cause the system to: obtain sequence data for a plurality of nucleic acid sequences present in a food product; identify one or more microbial signatures in the sequence data, wherein the one or more microbial signatures correspond to one or more microbes present in the food product; and authenticate a source for the food product by determining whether the one or more microbial signatures correspond to one or more microbial signatures associated with a particular source for the food product, wherein the food product is authenticated if the one or more microbial signatures correspond to the one or more microbial signatures associated with a particular source for the food product.

In another aspect, disclosed herein is a system for detecting the presence of a contaminant in a food production chain, comprising: one or more processors; and a memory comprising instructions executable by the one or more processors that, when executed by the one or more processors, cause the system to: obtain sequence data for a plurality of nucleic acid sequences present in a food product; identify one or more microbial signatures in the sequence data, wherein the one or more microbial signatures correspond to one or more microbes present in the food product; and detect a contaminant in the food product by: determining whether the one or more microbial signatures correspond to one or more microbial signatures associated with a particular contaminant, wherein the contaminant is detected if the one or more microbial signatures correspond to the one or more microbial signatures associated with a particular contaminant.

In another aspect, disclosed herein is a system for identifying a source for a food product comprising: one or more processors; and a memory comprising instructions executable by the one or more processors that, when executed by the one or more processors, cause the system to: obtain sequence data for a plurality of nucleic acid sequences present in a food product; identify one or more microbial signatures in the sequence data, wherein the one or more microbial signatures correspond to one or more microbes present in the food product; and identify a source for the food product by determining whether the one or more microbial signatures correspond to one or more microbial signatures associated with a particular source for a food product, wherein the source is identified if the one or more microbial signatures correspond to the one or more microbial signatures associated with a particular source for a food product.

### BRIEF DESCRIPTION OF THE FIGURES

FIG. 1 is a flow diagram depicting a method of authenticating a source for a food product.

FIG. 2 is a flow diagram depicting an exemplary data analysis process for the identification of a microbial signature for a food product. The dashed line indicates that microbial identification can be performed directly using a database of nucleic acid sequences corresponding to microbes associated with a particular food source.

FIG. 3 is an exemplary decision tree for authentication of a food source with one microbe.

FIG. 4 is a decision tree for a working example of food source authentication with one microbe.

FIG. 5 is an exemplary decision tree of food source authentication with multiple microbes associated with multiple food sources. The same food source is not considered at each stage. g, g-1, g-2 correspond to distinct food sources.

FIG. 6 is a decision tree for a working example of a food source authentication method with multiple microbes associated with multiple food sources. The same food sources are not considered at each stage. Instead of iterative reduction, a combined signature of presence/absence or relative levels of species can also be applied with the same process. Tables 1-5 show examples of databases of bacteria found in various materials.

### DETAILED DESCRIPTION

The following description sets forth exemplary methods, conditions, and the like and are not intended as limiting the scope of the present disclosure. Instead, it is provided as a description of exemplary embodiments.

### I. Overview

Disclosed herein are methods and systems for authenticating food products, identifying food sources, and/or detecting contaminants in a food product. The methods and systems disclosed herein are based on the analysis of microbial signatures corresponding to one or more microbes present in a food sample. Food products from different sources have different microbial signatures that can be used to identify the source for the food product, identify possible contaminants, and even identify a supply chain that was used to deliver the product.

Although the following description uses terms first, second, etc., to describe various elements, these elements should not be limited by the terms. These terms are only used to distinguish one element from another.

The terminology used in the description of the various embodiments described herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, rational numbers, steps, operations, elements, and/or components, but do not preclude the presence or addition of one or more other features, integers, rational numbers, steps, operations, elements, components, and/or groups thereof.

The term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

The terms “microbiome” or “microbiota” may be used with meanings and/or intention in the art, but as used in this specification, these terms may be construed to encompass any meaning and/or intention used by those in the art, unless otherwise specified.

Metagenomics generally relates to the study of genetic material that is obtained from an environment (e.g. a food product or food factory surface or food processing equipment surface) and allows for analysis of a sample without the need to isolate the genetic material from individual species present in the sample. Metagenomics allows food samples to be analyzed in an unbiased, high throughput, and comprehensive manner. However, most current nucleic acid-based methods for food authentication rely on authenticating sources of food raw materials by direct analysis of sequences corresponding to the raw materials, which can fail to properly authenticate food products if the sources are removed prior to authentication.

A given raw material can come from a plurality of different sources. For example, animal meal, such as chicken meal, may be sourced from different geographical regions, strains or breeds, feeding and housing conditions, etc. A particular food source can be associated with a particular microbiome composition and be used to identify a source for the food source.

A “microbiome” or “microbiota” generally relates to a community of microbes present in an environment. Shifts in the microbiome composition of a particular food product would reflect changes in the raw materials used for production of the food product. For instance, the presence of chicken from different geographical sources would lead to a change in the microbiome.

Furthermore, the presence of a pork source in a chicken product would lead to a change in the microbes present in the food source and could be an indication that there is contamination in the supply chain. This shift in the microbiome persists even if the contaminant is removed during a later step on food production. Thus, analysis of the microbes present in a particular food sample can be employed to indirectly identify and authenticate a food source, supply chain issues, and possible contamination within the supply chain.

The methods and systems disclosed herein provide for end-to-end trace-back for food source identification and authentication. Instead of relying on direct identification of food sources or raw materials, the methods described herein rely on the indirect authentication of food products based on the microbiome signatures that are present with the food products. Sequence data from nucleic acids present in food samples are analyzed to identify one or more microbial signatures. The microbial signatures can represent a subset of the metagenomics data obtained for a particular food product. The microbial signatures can be determined in a targeted manner by analyzing the data to identify a particular set of microbes associated with specific sources of interest. Alternatively, the sequence data can be analyzed in an unbiased manner to identify microbial signatures corresponding to detect deviations in the microbiome composition that can then be traced to particular sources. The identification of the microbial signatures and their use to authenticate a food source may not be dependent on the sequence data corresponding to the food raw materials themselves.

Furthermore, the methods disclosed herein may be used to trace-back the source of a food product to a particular supplier once the source has been identified or authenticated. For instance, if a preferred food source is identified, the testing producer can trace the source to a particular supplier. The producer can later use the methods disclosed herein to authenticate that the food product is sourced from the same food source based on microbes associated with the food source. Further, if an undesirable microbial signature or food source is identified, it can be traced back to a supplier, upon which the producer may seek corrective action or look to a different supplier.

Accordingly, the methods and system allow for improved accuracy and reliability in identifying and authenticating sources for food products.

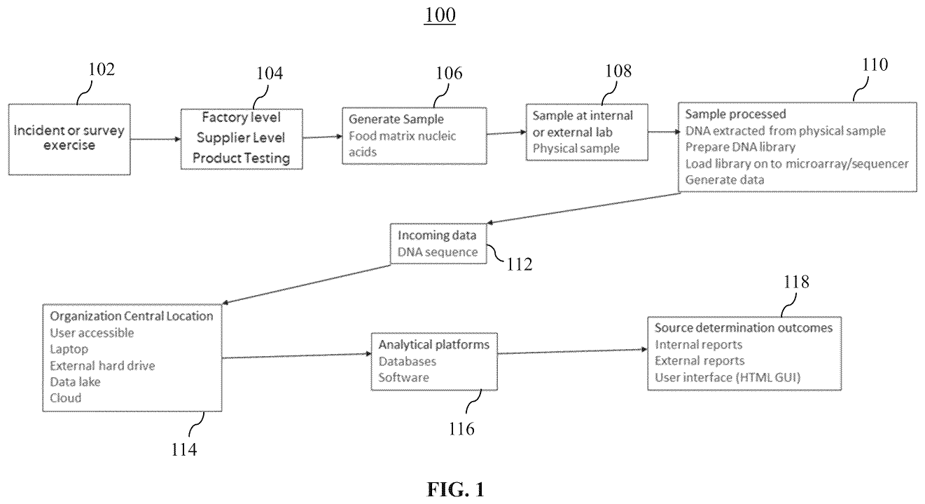

FIGS. 1-6 provide exemplary embodiments of methods for authenticating a food source for a food product, wherein sequence data for a food product is used to identify one or more microbial signatures that can be used to determine a food source based on the microbes present in the food product.

FIG. 1 depicts an exemplary flowchart of a method 100 for food authentication. At 102, the process may be initiated in response to an incident or survey exercise. The incident or survey exercise may be implemented as part of a regular monitoring process during any point of the food production line, or implemented to authenticate raw materials received from a new supplier. At 104, the incident or survey exercise prompts the implementation of the authentication method at, for example, the factory level, at the supplier level, or for product testing purposes. The method can be implemented at one or more particular points during each level of the food production chain. Moreover, at each level in the food production chain, one or more food products may be evaluated.

At 106, samples are generated from the food product at the designated testing level. The sample generation may involve preparation of food matrix nucleic acids. The sample is prepared at a designated location, such as an internal or external laboratory (108). Once the physical sample is received by the internal or external laboratory, the sample is processed at step 110. During sample processing, nucleic acids (e.g. DNA) are extracted from the physical sample, a sequencing library (e.g. a DNA library) is prepared, the library is analyzed (e.g. by loading the library onto a microarray or sequencer), and data is generated. Any known method for nucleic acid extraction and library preparation known in the art may be used. For instance, without limitation, nucleic acid extraction may be performed on freshly collected or frozen samples, and using any available extraction technique such as phenol:chloroform:isoamyl alcohol extraction or by using any appropriate commercially available kit. The sequencing library may be analyzed using any available technique that provides nucleic acid sequence data, such as, without limitation, next generation sequencing, qPCR, mass spectrometry, chromatography, microarray, in situ sequencing, probe hybridization, and any combination thereof. The sequencing library preparation will depend on the analysis technique to be used and can be prepared according to the manufacturer's instructions.

The generated data is transferred at 112 as incoming data (e.g., DNA sequence data) to a central location in the organization (114). The central location may be user accessible, such as a laptop, an external hard drive, a data lake or a cloud, or any other local or centralized system in the organization, or a data storage location available as a service to the organization. The transferred data may be provided from an internal laboratory or an external source, and may be stored in the central location until further downstream analysis. At 116, the data from the central location is accessed by analytical platforms. The analytical platform would comprise one or more databases or software that would enable analysis of the sequence data. Analysis of the nucleic acid sequences can include, without limitation, comparing sequences against one or more databases; filtering sequence reads by size, quality, or origin; de-multiplexing a sample; sequence mapping; read quantification; or any combination thereof. Any suitable analytical platform, such as a platform comprising publicly available software or database, or in-house software or databases may be used.

Analysis of the data using the analytical platforms results in one or more source determination outcomes (118). The one or more source determination outcomes may be included in internal or external reports, which may be reported as a physical report, or displayed in a user interface. For instance, a user interface may display the one or more source determination outcomes and allow a user to navigate and refine the outcomes. Additionally, the user interface may allow a user to compare one or more source determination outcomes corresponding to different food products, different lots of the same food products, the same product lot at different lots in the food production chain, or any combination thereof. In some instances, sequence data analysis may include determining one or more microbial signatures corresponding to one or more microbes in the food product.

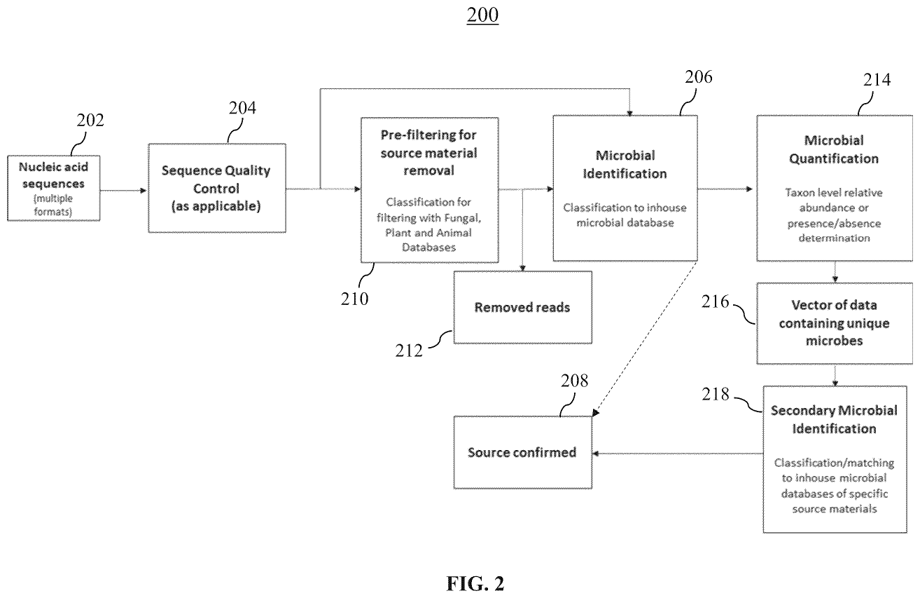

FIG. 2 depicts an exemplary analysis process for identifying one or more microbial signatures corresponding to one or more microbes in a food product (method 200). The one or more microbial signatures correspond to all microbes present in the food sample, or to a partial list. For example, the one or more microbial signatures may correspond to only one microbe present in the food product. Microbes known to be present in food include, without limitation, those belonging to the genus Parageobacillus, Blautia, Aliivibrio, Porphyrobacter, Shigella, Aneurinibacillus, Anaerostipes, Photobacterium, Erythrobacter, Rathayibacter, Butyrivibrio, Tyzzerella, Grimontia, Dechloromonas, Leifsonia, Coprothermobacter, Intestinimonas, Pseudoalteromonas, Pseudarthrobacter, Arthrobacter, Megasphaera, Ethanoligenens, Alteromonas, Isoptericola, Micrococcus, Eubacterium, Colwellia, Cellulomonas, Thermus, Oscillibacter, Yersinia, Nocardia, Meiothermus, Weissella, Edwardsiella, Gordonia, Rahnella, Murdochiella, Oceanimonas, Propionibacterium, Azotobacter, Eggerthella, Marinomonas, Tessaracoccus, Caulobacter, Adlercreutzia, Halomonas, Pimelobacter, Fibrobacter, Gordonibacter, Methylophaga, Actinoplanes, Fervidobacterium, Obesumbacterium, Brucella, Listeria, Methanobrevibacter, Plesiomonas, Caldanaerobacter, Deinococcus, Methanosarcina, Gallibacterium, Synechococcus, Spirosoma, Thioploca, Calothrix, Helicobacter, Thermotoga, Janthinobacterium, Nonlabens, Barnesiella, Fusobacterium, Ornithobacterium, Ilyobacter, Akkermansia, Thermodesulfobacterium, Cloacibacillus, Theileria, Gyrovirus, T7virus, T4virus, Alpharetrovirus, Sp18virus, Acidaminococcus, Altererythrobacter, Comamonas, Arcobacter, Aeromicrobium, Pediococcus, Proteus, Alistipes, Azospira, Geobacillus, Geoalkalibacter, Agrobacterium, Vibrio, Christensenella, Bosea, Kurthia, Hafnia, Alcaligenes, Clostridioides, Novosphingobium, Oblitimonas, Morganella, Amycolatopsis, Odoribacter, Pseudoxanthomonas, Negativicoccus, Aureimonas, Olsenella, Psychrobacter, Paenibacillus, Brachybacterium, Parabacteroides, Shewanella, Providencia, Brevibacterium, Roseburia, Candida, Ruminococcus, Caulimovirus, Selenomonas, Clavibacter, Treponema, Curtobacterium, Turicibacter, Erwinia, Frondihabitans, Hymenobacter, Kineococcus, Kluyveromyces, Massilia, Methylobacterium, Microbacterium, Nocardioides, Ochrobactrum, Pseudonocardia, Rhizobium, Saccharopolyspora, Sanguibacter, Shinella, Sphingobacterium, Sugiyamaella, Chryseobacterium, Aeromonas, Achromobacter, Blastomonas, Pantoea, Delftia, Anoxybacillus, Bordetella, Mycobacterium, Bacteroides, Brevundimonas, Rhodococcus, Bifidobacterium, Kosakonia, Streptomyces, Desulfovibrio, Sphingobium, Thermothelomyces, Flavonifractor, Sphingomonas, Thielavia, Lachnoclostridium, Sphingopyxis, Macrococcus, Cupriavidus, Moraxella, Prevotella, Ruminiclostridium, Bradyrhizobium, Campylobacter, Clostridium, Stenotrophomonas, Burkholderia, Cutibacterium, Xanthomonas, Serratia, Escherichia, Staphylococcus, Streptococcus, Variovorax, Acidovorax, Acinetobacter, Bacillus, Citrobacter, Corynebacterium, Enterobacter, Enterococcus, Klebsiella, Lactobacillus, Lactococcus, Pseudomonas, Raoultella, and Salmonella.

In an exemplary method to determine one or more microbial signatures, nucleic acid sequences are received at 202. The nucleic acid sequences may correspond to DNA, RNA, or both DNA and RNA sequences. The nucleic acid sequences may be provided in any suitable format. At 204, a sequence quality control may be implemented as applicable. The sequence quality control may include, without limitation, trimming, length filtering, sequencing adapter removal, sequence binning, or any combination thereof. The nucleic acid sequences are then analyzed for microbial identification (206), in which one or more microbial signatures corresponding to one or more microbes are identified. Microbial identification may include classification to a microbial database (e.g. an in-house microbial database). The microbial databases may be specific to a particular category of microbes, such as a viral database or a fungal database, or may correspond to a wide range of microbes. The microbial databases may also correspond to microbes present in a particular food source or set of food sources. Any suitable database corresponding to microbial sequences may be used for microbial identification. The databases may correspond to nucleic acid sequences consisting of combinations of nucleotides (e.g., A,T,G, or C), or they may correspond to amino acid sequences corresponding to one or more protein or protein isoforms encoded by microbial nucleic acids, and which may include any naturally and non-naturally occurring amino acid residues known in the art. After microbial identification, a source can be confirmed (208) based on the one or more microbial signatures. Prior to microbial identification, a pre-filtering step may be performed for removal of sequences corresponding to the source material (210). The pre-filtering step can include classification of sequences using fungal, plant, or animal databases. In some instances, pre-filtering can identify sequence reads that do not correspond to any microbe present in the food product (e.g. unmapped sequences). Pre-filtering can also be used to identify sequence reads corresponding to the food raw materials. At 212, the pre-filtered sequence reads may then be removed from the sequence data.

Microbial quantification (214) may also be performed. The microbial quantification may be determined as the taxon level relative abundance of one or more microbes, or as a presence or absence determination. Microbial quantification may be based on the number of reads corresponding to a particular microbe. For example, a higher read count for sequences corresponding to a particular microbe would indicate higher levels of that microbe in the food product. The quantification may be based on an internal or external control sample. In some instances, microbial quantification may include setting a threshold. For example, the presence or absence of a microbe may be determined based on whether sequence reads corresponding to that sample surpass a pre-determined threshold, such as, at least 90%, at least 95%, or a 100% sequence identity match to at least one sequence in a reference database. Following microbial quantification, a vector data containing unique microbes is generated (216). The vector data is used for secondary microbial identification at 218. Secondary microbial identification may include, for example, classification or matching of the microbial signatures to one or more microbial databases. For example, in-house microbial databases corresponding to specific source materials may be used for secondary microbial identification.

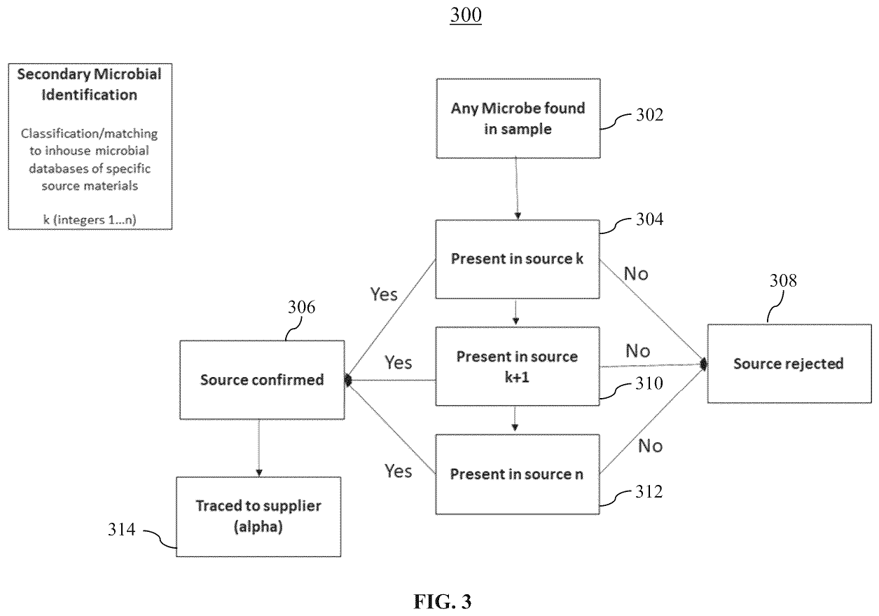

The methods described herein can comprise determining whether one or more microbial signatures correspond to one or more microbial signatures associated with a particular food source or a particular contaminant. FIG. 3 shows an exemplary process of determining whether microbial signatures correspond to one or more microbes associated with a particular food source (method 300). The method corresponds to secondary microbial classification and may involve classification or matching of microbial signatures to in-house microbial databases for specific source materials k (integers k+1 . . . n). The determination is performed as an iterative process, where only one food source is considered at each stage or step of the process. A microbial signature corresponding to any microbe found in the sample is received at 302. At 304, the microbial signature is analyzed to determine whether the microbe is present in food source k. If the microbe is present in food source k, the food source is confirmed at 306. Alternatively, if the microbe is not present in food source k, the food source is rejected (308). The analysis is iterated to determine whether the microbe is present in food source k+1. The food source is confirmed (306) if the microbe is present in food source k+1, but rejected (308) if the microbe is not present in food source k+1. The analysis is further iterated for food source n (312) with the source confirmed (306) if the microbe is present in food source n, but rejected (308) if the microbe is not present in food source n. Sources confirmed step 306 may be used for authentication of the food product. The confirmed source(s) are then be traced to a particular supplier (314).

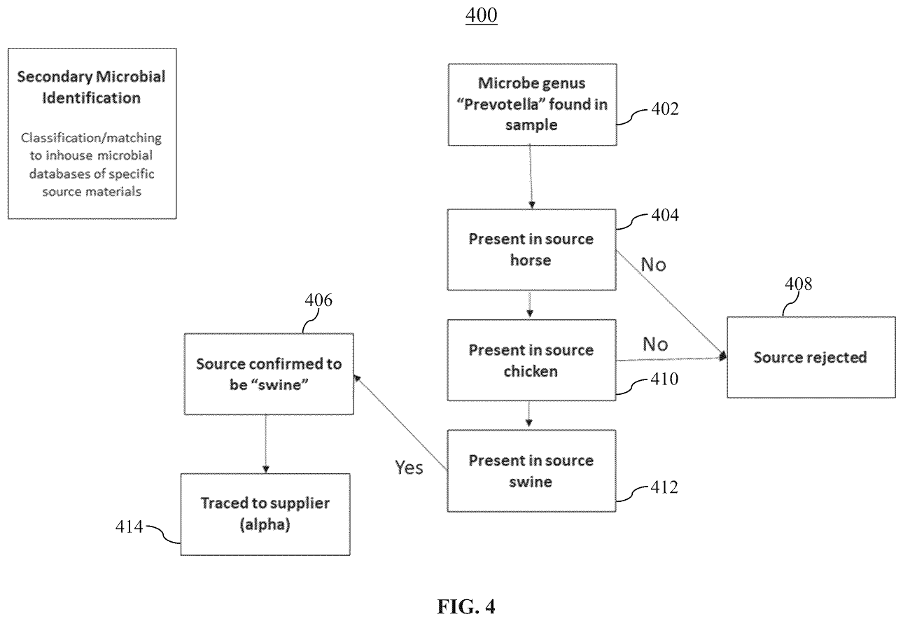

FIG. 4 depicts an example of secondary microbial identification for food authentication (method 400). Secondary microbial identification may be performed by classification or matching to in-house microbial databases of specific source materials. The determination is performed as an iterative process, where only one food source is considered at each stage or step of the process. In this example, a microbe belonging to the genus Prevotella is found in the sample (402). At 404, the microbial signature is analyzed to determine whether the microbe is present in a horse source. Horse is rejected as a source at step 406 based on the determination that microbes belonging the genus Prevotella are not present in the horse source. The microbial signature is then analyzed to determine whether the microbe is present in a second source corresponding to a chicken source (408). Chicken is rejected as a food source at 406 based on the determination that microbes belonging to the genus Prevotella are not present in a chicken source. At 410, the microbial signature is then analyzed to determine whether the microbe is present in a third source corresponding to a swine source. Based on the determination that microbes belonging to the genus Prevotella are present in a swine source, the food source is confirmed to be swine (412). The confirmed swine source can then be traced to a supplier at 414.

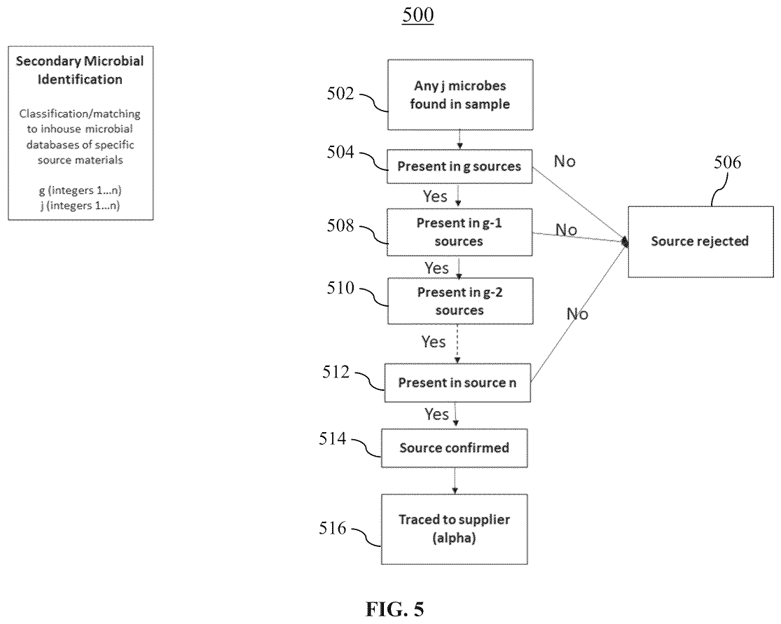

Multiple microbial signatures, each corresponding to one or more microbes present in the food product, can be analyzed to identify or authenticate a food source. FIG. 5 shows an exemplary method for secondary microbial identification for food source authentication using multiple microbial signatures correspond to one or more microbes associated with particular food sources (method 500). The method can involve classification or matching to in-house microbial databases of specific source materials. In this method, multiple microbial signatures are considered in parallel, but only one particular food source or contaminant is considered at each stage. At each stage, a food source is rejected as a possible source if the microbial signatures do not correspond to one or more microbes associated with the food source or contaminant being considered at that stage. The one or more microbes associated with each food source or contaminant g, g-1, g-2, etc., are different. The process may be repeated until all food sources or contaminants n have been considered. The determination may be performed in an iterative fashion, with a particular source considered at each different step of the process. Microbes corresponding to multiple sources may be considered first, or may be considered after microbes unique to a particular source have been first considered. Alternatively, the determination may be performed in parallel, with multiple sources being considered in parallel.

Microbial signatures corresponding to any j microbes present in the sample are received at step 502. At each of 504, 508, 510, and 512, the one or more microbial signatures obtained at 502 are analyzed to determine whether they correspond to any microbe j (integers 1 . . . n) associated with food source g (integers 1 . . . n). For instance, at 504, the microbial signature is analyzed to determine whether any j microbes are present in g sources. The g sources are rejected (506) if none of the j microbes are present in the g sources. Alternatively, the g sources are confirmed if one or more of the j microbes are present in the g sources. After 504, the one or more microbial signatures are analyzed to determine whether the any j microbes are present in g-1 sources (508). The g-1 sources are rejected at 506, if none of the j microbes are present in the g-1 sources. The g-1 sources are confirmed if one or more of the j microbes are present in the g-1 sources. Similarly, at 510, the microbial signature is analyzed to determine whether any j microbes are present in g-2 sources. The g-2 sources are confirmed if one or more of the j microbes are present in the g-2 sources. The process is iterated for n sources at 512, with the microbial signature analyzed to determine whether any j microbes are present in n sources. The n sources are rejected at step 506, if none of the j microbes are present in the n sources. If one or more of the j microbes are present in the n source, the n sources are confirmed. At 516, any confirmed source(s) are traced to a supplier.

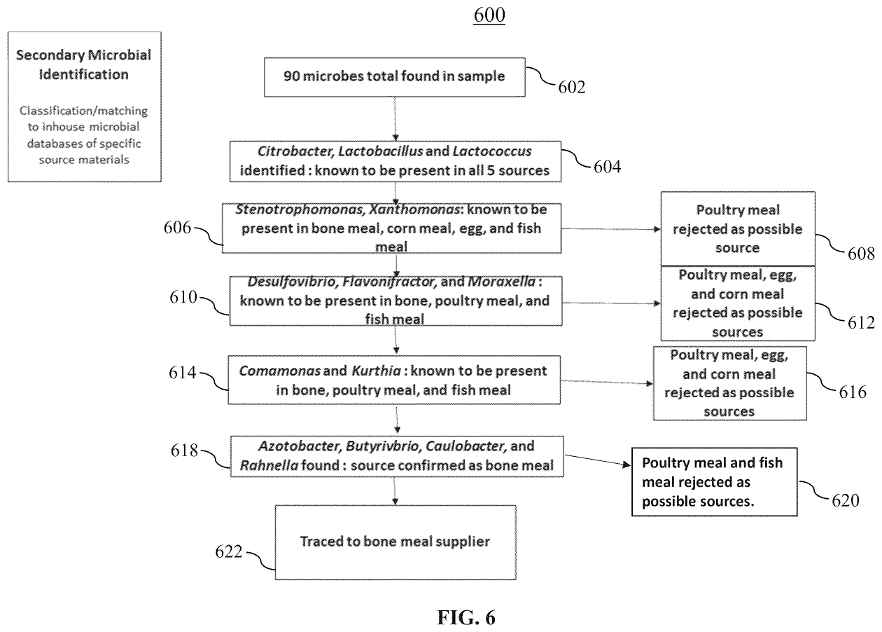

FIG. 6 shows an example of secondary microbial identification for food source authentication using multiple microbial signatures (method 600). The secondary microbial classification can be performed by classification or matching of microbial signatures to in-house microbial databases of specific source materials. Microbial signatures corresponding to 90 total microbes found in the food sample are received at 602. At 604, microbes belonging to the Citrobacter, Lactobacillus, and Lactococcus genus are identified. These microbes are known to present in all five sources analyzed in this exemplary embodiment (bone meal, corn meal, egg, fish meal, and poultry meal). At 606, microbes from the Stenotrophomonas and Xanthomonas genus are identified. These microbes are known to be present in bone meal, corn meal, egg, and fish meal sources, but known to not be present in chicken meal. Chicken meal is rejected as a possible source at 608. At 610, microbes belonging to the Desulfovibrio, Flavonifractor, and Moraxella genus are identified. Microbes from there genus are known to be present in bone meal, poultry meal, and fish meal. Poultry meal, egg, and corn meal are rejected as possible sources at step 612. At step 614, microbes belonging to the Comamonas and Kurthia genus are identified. These microbes are known to be present in bone meal, poultry meal, and fish meal. Poultry meal, egg, and corn meal are rejected as possible sources at 616. Microbes belonging to the Azotobacter, Butyrivbio, Caulobacter, and Rahnella genus are identified. Microbes from these genus are known to be present in bone meal. Accordingly, at step 618, the source is confirmed to be bone meal, while poultry meal, and fish meal are rejected as sources at 620. The source is then traced to a bone meal supplier at 622.

One or more different food sources may be authenticated or identified for a particular food product. The sources may correspond to known food sources for the food product, for example sources for on-label components, or to a preferred supplier for the raw materials. Authentication or identification of food sources may also be performed at multiple steps in the food production chain.

Similar methods as those disclosed herein may be used to detect the presence of a contaminating food source in a food product. The microbial signatures may correspond to microbes associated with a particular contaminant. The microbial signatures can then be analyzed by comparing to a database of microbes associated with particular contaminant food sources, thus allowing the detection of the contaminant.

A particular food source or contaminant may also be traced back to a particular supplier. After authentication or identification of the food source, the food source or contaminant may also be traced back to a particular supplier. For example, contamination of the food product during food production may be detected and traced back to a particular supplier. After analysis of microbial signatures from a chicken food product, a pork source may be identified during one point in the food production chain. The pork source may correspond, for instance, to a contaminating food source. The pork source can then be matched to a source provided by a particular supplier for that step of the food production chain. The supplier for the pork food source is then identified, and after some investigation, the supplier may find, for example, that the chicken raw materials were stored in the same transporting unit as pork raw materials. The presence of the pork source would lead to deviations in the microbiome of the food product that could be detected, even after the chicken source was no longer in close proximity to the food source. The food producer may consider that particular chicken source as compromised. The producer may then decide to implement a corrective action, such as issuing a warning to the supplier or changing to a different supplier altogether. In this way, the methods disclosed herein may be used for end-to-end trace-back of food sources or contaminants during the food production process. By authenticating the food sources at one or more steps of the food production chain, the producer can make sure that there is no adulteration introduced into the food product and certify its authenticity.

### II. Nucleic Acid Sequence Data

The methods disclosed herein comprise obtaining sequence data for a plurality of nucleic acid sequences present in a food product. The sequence data may correspond to any nucleic acid present in a food sample. For instance, the sequence data may correspond to a plurality of DNA and/or RNA sequences. The plurality of nucleic acid sequences may correspond to nucleic acids from one or more microbes present in food sample, or to nucleic acids from the food product.

Obtaining the sequence data can include extracting nucleic acids from the food product. Methods of extracting nucleic acids known in the art may be used. Without being limited, nucleic acids may be extracted using TrizolLS reagent, phenol:chloroform:isoamyl alcohol extraction, or equivalents. Nucleic acid extraction may also be performed using commercially available kits, such as, Ambion RNA isolation kits (e.g., Purelink RNA Mini kit or DynaBeads mRNA direct micro kit), MAgmax FFPE total nucleic acid isolation kit, Pall DNA and RNA Purification kits, Qiagen Allprep, PowerViral, Powersoil, or PowerMag kits, NEBNext Microbiome DNA Enrichment kit, or equivalents. Nucleic acid extraction may be performed using frozen or fresh samples. For example, a food product may be fixed before nucleic acid extraction. Nucleic acid extraction may also include a step of cell lysis. Cell lysis may be performed through any methods known to those skilled in the art, including, but not limited to, enzymatic lysis using lytic enzymes such as lysozyme, lysostaphin, mutanolysin, proteinase K, subtilisin, or any combination thereof; physical shearing, such as with glass beads, sonication, ultrasound, or high pressure; and any other cell lysis method known to those skilled in the art.

It should be understood that the present teachings contemplate sequence data that may be obtained using all available varieties of techniques, platforms, or technologies, including, but not limited to: capillary electrophoresis, microarrays, ligation-based systems, polymerase-based systems, hybridization-based systems, in situ sequencing, direct or indirect nucleotide identification systems, pyrosequencing, ion- or pH-based detection systems, electronic signature-based systems, etc.

The sequence data may be obtained by any method available in the art, such as by nucleic acid sequencing (e.g., next generation sequencing) or microarray analysis. The methods disclosed herein are not dependent upon a particular next sequencing technology, and the user needs to make appropriate choices for the intended downstream sequencing platform according to manufacturers' protocols. Exemplary sequencing platforms that may be used to obtain sequence data according to the methods disclosed herein include, but are not limited to, those produced by Illumina®, Oxford Nanopore™, Ion Torrent™, Roche™, Pacific Biosciences™, and Life Technologies™.

Depending on the sequencing technology used with the methods, a sequencing library may be prepared. The sequencing library will be representative of nucleic acids present in a food product and can be used with next generation sequencing platforms. Sequencing library preparation can include nucleic acid fragmentation, sample indexing, adaptor ligation, and library normalization. Sample indexing or barcoding allows multiple samples to be run simultaneously, taking full advantage of the high-throughput nature of current sequencing platforms. Adapter ligation is sequencing platform specific and standard to manufacturers' protocols. The adaptors may contain sequencing platform-specific end sequences and index sequences that allow for de-convolution of sequence data by sample. Barcoding and adapter ligation may be performed by any method known to those in the art, and may be adapted for analysis of the sequencing library with a particular sequencing platform. Library preparation can also include amplification, concentration, or dilution of the sequencing library. Libraries can be prepared at platform-specific concentrations of DNA and typically require amplification, concentration, or dilution to achieve the required concentration. The concentration of nucleic acids in the sequencing library may be determined by quantitative real-time PCR using platform specific manufacturer protocols or fluorescence-based measurement known in the art. In some instances, preparing the sequencing library includes selective enrichment of specific target nucleic acids or regions.

Nucleotide sequences of individual molecules are determined in a platform-specific manner to produce a raw dataset. The raw dataset can be converted to nucleotide sequencing information corresponding to each molecule in a sequencing library. The resulting products are whole “reads,” which may be processed to determine information about the food product. The sequence data may be produced in any format, such as BAM files, which are sequencing platform-independent and ready for bioinformatics analysis. Additional file types may include FASTA and FASTQ file formats, or other manufacturer-specific formats that can be converted to BAM, VCF, FASTQ, or FASTA format.

Once obtained, sequence data may be transferred in real time from the instrument used to generate sequence data as soon as the sequence data has reached a sufficient size in total base pairs for analysis, or it may be stored in a database until further analysis.

The sequence data may then be prepared for further analysis. This preparation can include performing sequence quality control, trimming, length filtering, sequencing adapter removal, and/or binning of reads by molecular barcode from the sequencing reads. In particular, the reads that represent the plurality of nucleic acid sequences from a food product can be quality controlled to remove the adapter sequences, clonal reads due to PCR amplification, and platform-specific sequence errors and filtered to achieve an acceptable error rate. Sequencing reads in the sequencing data may be deconstructed into, for example, k-mers of a particular size. Sequence assembly, mapping, or pairwise comparison of the sequencing reads in the sequence data may also be performed. In some cases, nucleic acid sequences corresponding to the food product or another agent can be filtered or removed from the sequence data prior to further analysis.

### III. Microbial Signatures

The methods described herein can include identifying one or more microbial signatures in the sequence data. The one or more microbial signatures may correspond to one or more microbes present in the food product. The microbial signatures may correspond to a particular taxonomic level, such as a kingdom, a phylum, a class, a genus, a species, a serotype or a strain. The microbes present in the food source may be microbial eukaryotes, bacteria, archaea, fungi, or viruses.

Identifying the one or more microbial signatures can include comparing sequence data to one or more databases. The databases can contain sequences (e.g., nucleic acid sequences, or amino acid sequences) from a particular group of microbes. For instance, the databases may correspond to nucleic acid sequences from microbes associated with particular food sources, or particular contaminants. These databases may correspond to a specific microbial taxa, a specific genus, or a collection of microbial species. Any publicly available database that is suitable for microbe identification may be used. Alternatively, an in-house database may be generated and used to identify a microbial signature.

Identifying one or more microbial signatures can include determining the relative level of nucleic acids in the sequence data corresponding to a particular microbe in the food product. The relative level of the nucleic acids in the microbial signature can be indicative of the relative level of the microbes and may be used to determine the relative abundance of a food source or contaminant in a food product. A threshold may be set, and any microbial signature corresponding to a relative level of microbes above the predetermined threshold would be indicative of the presence of the particular food source or contaminant. The threshold may be set in terms of, for example, a Ct value, a nucleic acid copy number, a concentration (e.g., in mg/mL or mg/L units), etc.

The microbes present in the food sample may be associated with a particular food source or contaminant. The food source may be of fungal, plant, or animal origin. Sources of plant origin can include, but are not limited to, corn, rice, wheat, soy, peanut, tree nuts, or any variety thereof, or any other plant sources, such as domesticated or wild plant crops, that may be used in food production. Corn meal is a commonly used food source of plant origin. Sources of fungal origin may correspond to a yeast, a mold, or a mushroom. Without limitation, food sources of animal origin may be from fish, beef, goat, egg, pork, poultry, or shellfish. The animal origin may correspond to various meats, such as canine, feline, equine, bovine, or ovine meats, or meat from any other source, such as from a game animal. Egg, poultry meal, fish meal, and bone meal are examples of commonly used raw materials of animal origin. Any other animal food source suitable for food production may also be identified.

In some instances, the one or more microbial signatures correspond to one or more microbes associated with a particular geographical region. Without being limited by theory, a particular microbe may be present in a particular region due to the presence of certain favorable conditions (e.g. soil composition, climate, temperature, altitude, topography, human management, etc.). However, the same microbe may be absent or rarely observed outside of that geographical region, and thus will be detected mainly in food sources from that geographical region. For example, a particular microbe or a particular species of this microbe may be detected in a plant cultivar from one particular region but will not be detected from a similar cultivar in a different region. The presence of the microbe or particular species in a food product would indicate that the raw materials were sourced from the particular region were the microbe can be found. The geographical region may be limited to a particular farm or field, or may extend to a bigger geographical are sharing the same topology and climate.

Microbial signatures may correspond to one or more of a bacteria, virus, archaea, or eukaryotic microorganisms. Exemplary microbes that can be present in food include, but are not limited to, microbes belonging to a genus taxonomy selected from the group consisting of Parageobacillus, Blautia, Aliivibrio, Porphyrobacter, Shigella, Aneurinibacillus, Anaerostipes, Photobacterium, Erythrobacter, Rathayibacter, Butyrivibrio, Tyzzerella, Grimontia, Dechloromonas, Leifsonia, Coprothermobacter, Intestinimonas, Pseudoalteromonas, Pseudarthrobacter, Arthrobacter, Megasphaera, Ethanoligenens, Alteromonas, Isoptericola, Micrococcus, Eubacterium, Colwellia, Cellulomonas, Thermus, Oscillibacter, Yersinia, Nocardia, Meiothermus, Weissella, Edwardsiella, Gordonia, Rahnella, Murdochiella, Oceanimonas, Propionibacterium, Azotobacter, Eggerthella, Marinomonas, Tessaracoccus, Caulobacter, Adlercreutzia, Halomonas, Pimelobacter, Fibrobacter, Gordonibacter, Methylophaga, Actinoplanes, Fervidobacterium, Obesumbacterium, Brucella, Listeria, Methanobrevibacter, Plesiomonas, Caldanaerobacter, Deinococcus, Methanosarcina, Gallibacterium, Synechococcus, Spirosoma, Thioploca, Calothrix, Helicobacter, Thermotoga, Janthinobacterium, Nonlabens, Barnesiella, Fusobacterium, Ornithobacterium, Ilyobacter, Akkermansia, Thermodesulfobacterium, Cloacibacillus, Theileria, Gyrovirus, T7virus, T4virus, Alpharetrovirus, Sp18virus, Acidaminococcus, Altererythrobacter, Comamonas, Arcobacter, Aeromicrobium, Pediococcus, Proteus, Alistipes, Azospira, Geobacillus, Geoalkalibacter, Agrobacterium, Vibrio, Christensenella, Bosea, Kurthia, Hafnia, Alcaligenes, Clostridioides, Novosphingobium, Oblitimonas, Morganella, Amycolatopsis, Odoribacter, Pseudoxanthomonas, Negativicoccus, Aureimonas, Olsenella, Psychrobacter, Paenibacillus, Brachybacterium, Parabacteroides, Shewanella, Providencia, Brevibacterium, Roseburia, Candida, Ruminococcus, Caulimovirus, Selenomonas, Clavibacter, Treponema, Curtobacterium, Turicibacter, Erwinia, Frondihabitans, Hymenobacter, Kineococcus, Kluyveromyces, Massilia, Methylobacterium, Microbacterium, Nocardioides, Ochrobactrum, Pseudonocardia, Rhizobium, Saccharopolyspora, Sanguibacter, Shinella, Sphingobacterium, Sugiyamaella, Chryseobacterium, Aeromonas, Achromobacter, Blastomonas, Pantoea, Delftia, Anoxybacillus, Bordetella, Mycobacterium, Bacteroides, Brevundimonas, Rhodococcus, Bifidobacterium, Kosakonia, Streptomyces, Desulfovibrio, Sphingobium, Thermothelomyces, Flavonifractor, Sphingomonas, Thielavia, Lachnoclostridium, Sphingopyxis, Macrococcus, Cupriavidus, Moraxella, Prevotella, Ruminiclostridium, Bradyrhizobium, Campylobacter, Clostridium, Stenotrophomonas, Burkholderia, Cutibacterium, Xanthomonas, Serratia, Escherichia, Staphylococcus, Streptococcus, Variovorax, Acidovorax, Acinetobacter, Bacillus, Citrobacter, Corynebacterium, Enterobacter, Enterococcus, Klebsiella, Lactobacillus, Lactococcus, Pseudomonas, Raoultella, and Salmonella.

The microbial signatures can correspond to one or more microbes associated with one food source or contaminant. Microbes associated with one food source or contaminant include, but are not limited to, the microbes listed in Table 1 of the examples. In some instances, the microbial signatures can correspond to one or more microbes associated with two or more food sources or contaminants. Without limitation, microbes associated with two or more food sources or contaminants include those listed in Tables 2-5 of the examples. The microbes may be associated with at least two food sources or contaminants, or they may be associated with more than two food sources, such as three or four food sources or contaminants.

### IV. Systems

In one aspect, the disclosure provides systems for performing any of the methods described herein.

The system can be configured to authenticating a source for a food product. For example, the system may include one or more processors and a memory comprising instructions executable by the one or more processors. When executed by the one or more processors, the instructions may cause the system to obtain sequence data for nucleic acid sequences present in a food product; identify one or more microbial signatures in the sequence data; and authenticate a source for the food product. The system may also be configured to determine whether the one or more microbial signatures correspond to microbes associated with a particular source, and authenticate the food source if the microbial signatures correspond to microbes associated with a known source for the product.

The system may also be configured for identifying a food source for the food product, or for detecting the presence of a contaminant in the food product.

### V. Methods in Computer-Readable Storage Devices

Any of the methods described herein can be implemented by computer-executable instructions or code stored in one or more computer-readable medium (e.g., a memory, a magnetic storage, an optical storage, or the like). Such instructions can cause one or more processors to implement the method.

### EXAMPLES

### Example 1

This example describes a microbial source end-to-end trace-back method for food source authentication. The method utilizes microbiome data from raw materials to authenticate the contents of the raw materials by using data from high throughput sequencing of nucleic acids present in a food sample.

FIG. 1 shows an overview of a method for food source authentication. Samples are collected at any identifiable part of the food manufacturing process in a facility and may include raw materials or finished products of any nature. Nucleic acids (DNA or RNA) are then extracted using commercial kits or using other approaches, such as phenol-chloroform-isoamyl alcohol reagents, Trizol LS, or other reagents that involve the use of guanidium thiocyanate combined with phenol. The extracted nucleic acids are preserved via protective agents such as RNALater, beta-mercaptoethanol, or equivalent chemical reagents. The nucleic acids are used immediately or stored for later analysis. In the case of RNA samples, the sample is reverse-transcribed prior to further use. The extracted nucleic acids can undergo selective amplification to enrich for specific regions or can undergo genomic scale amplification for further analyses. A sequencing library is then prepared by one or more means by commercial kits or a collation of specific reagents to prepare a sequencing library. The sequencing library is then applied to a chip in for sequencing using a high throughput sequencer, such as the Illumina, Oxford Nanopore, ThermoFisher's Ion Torrent, PacBio platforms, or equivalents.

Alternately, the nucleic acids are prepared for microarray applications by shearing and molecular detection reagent labelling for fluorescent or chemiluminescent detection. Mass spectrometry, capillary gel electrophoresis, and high performance liquid chromatography can also be used to generate specific patterns of nucleic acid contents. Subsequent detailed analysis of specific molecules, either from the output of the separation technologies, from microarray, or from other solid surface capture techniques, is performed. Any of these methods can be used in combination to generate nucleic acid sequence data, with or without using high throughput sequencing.

The sequence data is then analyzed with the help of bioinformatics by applying several steps, including data quality checking and filtering, creating databases of nucleic acid sequences corresponding to known species, removing any specific parts of the data that are not of interest such as other eukaryotes or specific microbial species, and then identifying a microbial signature from the sequence data. FIG. 2 depicts an overview of the sequence data analysis process.

The identified microbial signature is matched with known microbial signatures of specific sources to determine sources of microbes. Any microbes unique to a specific source can confirm specific raw material contamination (FIG. 3). For example, the presence of swine-specific bacterial species in poultry meal would indicate swine meat contamination (FIG. 4). Authentication can also be performed using microbial signatures corresponding to multiple microbes associated with more than one source (FIG. 5 and FIG. 6). A food source many also be authenticated without fully identifying all microbes present in a food product. Microbial genus associated with particular food sources are summarized in Tables 1-5.

TABLE 1
Bacteria signatures at genus taxonomy level unique to various food raw materials.

| Unique to bone | Unique to | Unique to fish | | Unique to |
|---|---|---|---|---|
| meal | poultry meal | meal | Unique to egg | corn powder |
| Parageobacillus | Blautia | Aliivibrio | Porphyrobacter | Shigella |
| Aneurinibacillus | Anaerostipes | Photobacterium | Erythrobacter | Rathayibacter |
| Butyrivibrio | Tyzzerella | Grimontia | Dechloromonas | Leifsonia |
| Coprothermobacter | Intestinimonas | Pseudoalteromonas | Pseudarthrobacter | Arthrobacter |
| Megasphaera | Ethanoligenens | Alteromonas | | Isoptericola |
| Micrococcus | Eubacterium | Colwellia | | Cellulomonas |
| Thermus | Oscillibacter | Yersinia | | Nocardia |
| Meiothermus | Weissella | Edwardsiella | | Gordonia |
| Rahnella | Murdochiella | Oceanimonas | | Propionibacterium |
| Azotobacter | Eggerthella | Marinomonas | | Tessaracoccus |
| Caulobacter | Adlercreutzia | Halomonas | | Pimelobacter |
| Fibrobacter | Gordonibacter | Methylophaga | | Actinoplanes |
| Fervidobacterium | Obesumbacterium | Brucella | | Listeria |
| Methanobrevibacter | Plesiomonas | Caldanaerobacter | | Deinococcus |
| Methanosarcina | Gallibacterium | Synechococcus | | Spirosoma |
| | Thioploca | Calothrix | | |
| | Helicobacter | Thermotoga | | |
| | Janthinobacterium | Nonlabens | | |
| | Barnesiella | Fusobacterium | | |
| | Ornithobacterium | Ilyobacter | | |
| | Akkermansia | Thermodesulfobacterium | | |
| | Cloacibacillus | Theileria | | |
| | Gyrovirus | T7virus | | |
| | T4virus | Alpharetrovirus | | |
| | Sp18virus | | | |

TABLE 2
Bacteria signatures at genus taxonomy level common to any two food raw materials

| Bone | Bone | Bone | Poultry | Corn | Corn | Egg |
|---|---|---|---|---|---|---|
| and Poultry | and Egg | and Fish | and Fish | and Egg | and Fish | and Fish |
| Acidaminococcus | Altererythrobacter | Comamonas | Arcobacter | Aeromicrobium | Pediococcus | Proteus |
| Alistipes | Azospira | Geobacillus | Geoalkalibacter | Agrobacterium | Vibrio | |
| Christensenella | Bosea | Kurthia | Hafnia | Alcaligenes | | |
| Clostridioides | Novosphingobium | Oblitimonas | Morganella | Amycolatopsis | | |
| Odoribacter | | Pseudoxanthomonas | Negativicoccus | Aureimonas | | |
| Olsenella | | Psychrobacter | Paenibacillus | Brachybacterium | | |
| Parabacteroides | | Shewanella | Providencia | Brevibacterium | | |
| Roseburia | | | | Candida | | |
| Ruminococcus | | | | Caulimovirus | | |
| Selenomonas | | | | Clavibacter | | |
| Treponema | | | | Curtobacterium | | |
| Turicibacter | | | | Erwinia | | |
| | | | | Frondihabitans | | |
| | | | | Hymenobacter | | |
| | | | | Kineococcus | | |
| | | | | Kluyveromyces | | |
| | | | | Massilia | | |
| | | | | Methylobacterium | | |
| | | | | Microbacterium | | |
| | | | | Nocardioides | | |
| | | | | Ochrobactrum | | |
| | | | | Pseudonocardia | | |
| | | | | Rhizobium | | |
| | | | | Saccharopolyspora | | |
| | | | | Sanguibacter | | |
| | | | | Shinella | | |
| | | | | Sphingobacterium | | |
| | | | | Sugiyamaella | | |

TABLE 3
Bacteria signatures at genus taxonomy level common to any three food raw materials.

| Bone, Chicken | Bone, Chicken | Bone, Corn | Bone, Egg | Chicken, | Corn, Egg and |
|---|---|---|---|---|---|
| and Corn | and Fish | and Egg | and Fish | Corn and Egg | Fish |
| Chryseobacterium | Aeromonas | Achromobacter | Blastomonas | Pantoea | Delftia |
| | Anoxybacillus | Bordetella | | | Mycobacterium |
| | Bacteroides | Brevundimonas | | | Rhodococcus |
| | Bifidobacterium | Kosakonia | | | Streptomyces |
| | Desulfovibrio | Sphingobium | | | Thermothelomyces |
| | Flavonifractor | Sphingomonas | | | Thielavia |
| | Lachnoclostridium | Sphingopyxis | | | |
| | Macrococcus | Cupriavidus | | | |
| | Moraxella | | | | |
| | Prevotella | | | | |
| | Ruminiclostridium | | | | |

TABLE 4
Bacteria signatures at genus taxonomy level common to any four food raw materials.

| Bone, Chicken, | Bone, Chicken, | Bone, Chicken, | Bone, Corn, Egg, | Chicken, Corn, |
|---|---|---|---|---|
| Corn, and Egg | Corn, and Fish | Egg, and Fish | and Fish | Egg, and Fish |
| Bradyrhizobium | Campylobacter | Clostridium | Stenotrophomonas | Burkholderia |
| | Cutibacterium | | Xanthomonas | Serratia |
| | Escherichia | | | Staphylococcus |
| | Streptococcus | | | Variovorax |

TABLE 5
Bacteria signatures at genus taxonomy
level common to all five food raw materials.
Bone, Chicken, Corn, Fish and Egg

Acidovorax
Acinetobacter
Bacillus
Citrobacter
Corynebacterium
Enterobacter
Enterococcus
Klebsiella
Lactobacillus
Lactococcus
Pseudomonas
Raoultella
Salmonella

### Example 2

This example describes the use of metagenomics filtering and microbial signatures for authenticate food samples.

### Materials and Methods

Identification of microbial signatures from sequence data can be performed as described below, or as described in Beck, K. L., et al. (2021) NPJ Sci Food, 5(1):3. The steps are modified as required by the particular platform employed to obtain the sequence data. Equivalent methods can be used to obtain information on the presence or absence, or of relative levels, of microbial species based on analyses of any DNA or RNA sequences.

### Sample Collection, Preparation, and Sequencing

Animal meal, corn meal, or egg powder samples were collected from a local market in the United States. Sample preparation, total RNA extraction and integrity confirmation, cDNA construction, and library construction for these samples was previously described by Haiminen, N., et al. (2019) NPJ Sci Food 3:24.

The samples were used to extract total RNA as described by Chen, P., et al. (2017) Pathogens 6:68, and total DNA as described elsewhere (Weis, A. M., et al. (2016). Appl. Environ. Microbiol 82:7165-7175; Emond-Rheault, J.-G., et al. (2017) Front. Microbiol. 8:996; Miller, B., et al. (2015) Kapa Biosyst. Appl. Note 1-8 (2015); Lüdeke, C. H. M., et al. (2015) Genome Announc. 3:2-3; Jeannotte, R., et al. (2015) Agil. Appl. Note 1-8; Arabyan, N., et al. (2016) Sci Rep 6: 29525). DNA and RNA purity (A_{260/230} and A_{260/280} ratios ≥1.8) and integrity were confirmed with Nanodrop (Nanodrop Technologies, Wilmington, DE, USA) and BioAnalyzer RNA Kit (Agilent Technologies Inc., Santa Clara, CA, USA) (Chen, P., et al. (2017) Pathogens 6:68). Subsequently, wherever RNA was used, cDNA was constructed using RNA (4 to 15 μg total input) and the SuperScript Double Stranded cDNA Synthesis kit (Invitrogen, Catalog no. 11917-020, Life Technology Carlsbad, CA). DNA processing does not require this particular step.

Sequencing libraries using HyperPrep Plus (Kapa BioSystems, Wilmington, MA, USA) cDNA/DNA were constructed as described previously (Chen, P., et al. (2017) Pathogens 6:68; Chen, P., et al. (2017) Appl Env. Microbiol 83; Kol, A., et al. (2014) Stem Cells Dev 23: 1831-1843) with an insert size between 300-400 bp. Library quantification was performed using qPCR (Library Quantification kit, catalog no. KK4824, Illumina, San Diego, CA) prior to submission for sequencing. The Illumina HiSeq 4000 (San Diego, CA) was used with 150 paired-end chemistry for each sample except the following: HiSeq 2000 with 100 paired-end chemistry was used for the four preliminary samples, and HiSeq 3000 with 150 paired-end chemistry was used for two other samples (MFMB-04 and MFMB-17).

### Sequence Data Quality Control

Illumina Universal adapters were removed and reads were trimmed using Trim Galore (Morgulis, A., et al. (2006) J. Comput. Biol. 13:1028-1040) with a minimum read length parameter 50 basepairs (bp). The resulting reads were filtered using Kraken software as described below with a custom database built from the PhiX genome (NCBI Reference Sequence: NC_001422.1). Trimmed non-PhiX reads were used in subsequent matrix filtering and microbial identification steps.

### Matrix Filtering Process and Validation

Kraken (Wood, D. E., and Salzberg, S. L. (2014) Genome Biol. 15:R46), with a k-mer size of 31 bp, was used to identify and remove reads that matched a pre-determined list of 31 common food matrix and potential contaminant eukaryotic genomes. These food matrix organisms were chosen based on preliminary eukaryotic read alignment experiments of the poultry meal samples as well as high-volume food components in the supply chain. Due to the large size of eukaryotic genomes in the custom Kraken database, a random k-mer reduction was applied to reduce the size of the database by 58% (using Kraken-build with option “--max-db-size”), in order to fit the database in 188 GB for in-memory processing. A conservative Kraken score threshold of 0.1 was applied to avoid filtering microbial reads. The matrix-filtering database includes low complexity and repeat regions of eukaryotic genomes to capture all possible matrix reads. This filtering database and the score threshold were also used in the matrix filtering for in silico testing as described below.

### Microbial Identification.

Remaining reads after quality control and matrix filtering were classified using Kraken (Wood, D. E., and Salzberg, S. L. (2014) Genome Biol. 15:R46) against a microbial database with a k-mer size of 31 bp to determine the microbial composition within each sample. NCBI RefSeq complete genomes were obtained for bacterial, archaeal, viral, and eukaryotic microorganisms (˜7,800 genomes retrieved on April 2017). Low complexity regions of the genomes were masked using Dustmasker (Morgulis, A., et al. (2006) J. Comput. Biol. 13:1028-1040) with default parameters. A threshold of 0.05 was applied to the Kraken score in an effort to maximize the F-score of the result. Taxa-specific sequence reads were used to identify presence or absence of microbial species, with a minimum of 10 reads required as the threshold for positive presence determination.

1. A method for authenticating a source for a food product comprising:
obtaining sequence data for a plurality of nucleic acid sequences present in a food product;
identifying one or more microbial signatures in the sequence data, wherein the one or more microbial signatures correspond to one or more microbes present in the food product; and
authenticating a source for the food product by:
determining whether the one or more microbial signatures correspond to one or more microbial signatures associated with a particular source for the food product,
wherein the food product is authenticated if the one or more microbial signatures correspond to the one or more microbial signatures associated with a particular source for the food product.

2. (canceled)

3. A method for detecting a contaminant in a food production chain, comprising:
obtaining sequence data for a plurality of nucleic acid sequences present in a food product;
identifying one or more microbial signatures in the sequence data, wherein the one or more microbial signatures correspond to one or more microbes present in the food product; and
detecting a contaminant in the food product by:
determining whether the one or more microbial signatures correspond to one or more microbial signatures associated with a particular contaminant,
wherein the contaminant is detected if the one or more microbial signatures correspond to the one or more microbial signatures associated with a particular contaminant.

4. The method of claim 1, wherein the one or more microbial signatures correspond to the genus taxonomy of one or more microbes present in the food product.

5. The method of claim 1, wherein the one or more microbial signatures correspond to the species or serotype taxonomy of one or more microbes present in the food product.

6. The method of claim 1, wherein the one or more microbial signatures corresponds to the relative level of the one or more microbes present in the food product.

7. The method of claim 1, wherein the one or more microbial signatures correspond to one or more microbes associated with a single source or a single contaminant.

8. The method of claim 1, wherein the one or more microbial signatures correspond to one or more microbes associated with two or more sources or two or more contaminants.

9. The method of claim 1, wherein the source or the contaminant is of animal origin.

10. (canceled)

11. The method of claim 1, wherein the source or the contaminant is of plant or fungal origin.

12. (canceled)

13. The method of claim 1, wherein the source or the contaminant corresponds to a particular geographical region.

14. The method of claim 1, wherein the one or more microbes are selected from the group consisting of bacteria, viruses, archaea, and eukaryotic microorganisms.

15. (canceled)

16. The method of claim 1, wherein obtaining the sequence data comprises preparing a sequencing library.

17. The method of claim 1, wherein obtaining the sequence data comprises next generation sequencing, or microarray analysis.

18. The method of claim 1, wherein the plurality of nucleic acid sequences are DNA sequences.

19. The method of claim 1, wherein the plurality of nucleic acid sequences are RNA sequences.

20. The method of claim 1, wherein sequences corresponding to the food product are filtered from the sequence data prior to identifying the one or more microbial signatures.

21. The method of claim 1, wherein identifying the one or more microbial signatures comprises comparing the sequence data against one or more databases of microbial nucleic acid sequences.

22. The method of claim 1, wherein determining whether the one or more microbial signatures correspond to one or more microbial signatures associated with a particular source for the food product comprises comparing the one or more microbial signatures against one or more databases of microbial signatures associated with the particular source; or wherein determining whether the one or more microbial signatures correspond to one or more microbial signatures associated with a particular contaminant comprises comparing the one or more microbial signatures against one or more databases of microbial signatures associated with the particular contaminant.

23. The method of claim 1, wherein the method is performed at two or more points in a food production chain of the food product.

24-26. (canceled)

27. A system for identifying a source for a food product comprising:
one or more processors; and
a memory comprising instructions executable by the one or more processors that, when executed by the one or more processors, causes the system to:
obtain sequence data for a plurality of nucleic acid sequences present in a food product;
identify one or more microbial signatures in the sequence data, wherein the one or more microbial signatures correspond to one or more microbes present in the food product; and
identify a source for the food product by:
determining whether the one or more microbial signatures correspond to one or more microbial signatures associated with a particular source for a food product,
wherein the source is identified if the one or more microbial signatures correspond to the one or more microbial signatures associated with a particular source for a food product.
